(19) United States
(12) Patent Application Publication
(10) Pub. No.: US 2006/0019929 A1
(43) Pub. Date: Jan. 26, 2006

(54) Combination therapies employing platelet aggregation drugs

(76) Inventor: Albert Friesen, Winnipeg (CA)

Correspondence Address: MERCHANT & GOULD PC, P.O. BOX 2903, MINNEAPOLIS, MN, 55402-0903, US

(21) Appl. No.: 11/177,248

(22) Filed: Jul. 7, 2005

Related U.S. Application Data
(60) Provisional application No. 60/585,577, filed on Jul. 7, 2004.

(51) Int. Cl. A61K 31/675; A61K 31/4415

(52) U.S. Cl. 514/89; 514/350

(57) ABSTRACT

The present invention provides pharmaceutical compositions comprising a platelet aggregation inhibitor and a compound selected from pyridoxal-5′-phosphate, a pyridoxal-5′-phosphate related compound, or a pharmaceutically acceptable salt thereof. The invention also includes methods for using a platelet aggregation inhibitor and a compound selected from pyridoxal-5′-phosphate, a pyridoxal-5′-phosphate related compound, or a pharmaceutically acceptable salt thereof.

This application claims the benefit under 35 U.S.C. §119(e) of U.S. provisional application Ser. No. 60/585,577, filed on Jul. 7, 2004, the disclosure of which is incorporated by reference.

### FIELD OF INVENTION

The present invention relates to pharmaceutical compositions and uses thereof for treatment of cardiovascular disease, in particular the present invention relates to the use of combination therapies employing platelet aggregation drugs.

### BACKGROUND

The role of platelets in the pathophyisology of atheroscelerotic disease and thrombotic events is well known. Long term prophylatic use of antiplatelet drugs, which inhibit platelet aggregation, has been shown to be beneficial in the prevention of ischemic stroke, myocardial infarction, unstable angina, peripheral arterial disease, need for vascular bypass or angioplasty, and vascular death in patients at increase risk of such outcomes, including those with established atherosclerosis or a history of atherothrombosis.

Currently there are numerous antiplatelet drugs which are widely available and combination therapies have been and continued to be investigated. Most antiplatelet drugs have side effects, and increasing the dosage leads to increased side effects. Thus, combination therapies have been tried. However, many combination therapies are ineffective for various reasons. For example, many drugs are contraindicated. In other cases, drugs work through mechanisms of action (sometimes unknown) that result in a lack of synergy for attempted combinations.

The present inventors have found that pyridoxal-5-phosphate (P5P) and certain P5P related compounds, which also have antithrombotic properties, are well tolerated drugs with no significant side effects. Furthermore, P5P and P5P related compounds positively modulate multiple cardiovascular risk factors including lipoprotein and homocysteine levels. Previous disclosures have taught the use of vitamin B6 (pyroxdine) with an antiplatelet agent wherein the inclusion of vitamin B6 was directed to decreasing homocysteine levels. For example, U.S. Pat. No. 6,323,188 discloses a method of reducing the incidence and severity of stroke, primary heart attack and any subsequent stroke or heart attack comprising the daily administration of acetylsalicylic acid (ASA), a vitamin B12 compound, a folic acid compound, and vitamin B6. U.S. Pat. No. 6,121,249 discloses a method reducing the incidence and severity of atherosclerosis, atherosclerotic central nervous system disease, claudication, coronary artery disease, homocysteine related disorders, hypertension, peripheral vascular disease, presenile dementia, and/or restenosis comprising daily administration of ASA, a vitamin B12 compound, a folic acid compound, and vitamin B6. U.S. Pat. No. 6,274,170 discloses compounds for the treatment of atherosclerotic cardiovascular disease comprising ASA, ascorbic acid, folic acid, vitamin E, vitamin B6, and vitamin B12. However, there are currently no combination therapies which employ a pyridoxal-5′-phosphate or pyridoxal-5′-phosphate related compound with an antiplatelet agent.

### SUMMARY OF INVENTION

In a first aspect, the present invention provides a novel pharmaceutical composition comprising: (a) a compound selected from pyridoxal-5′-phosphate, a pyridoxal-5′-phosphate related compound, or a pharmaceutically acceptable salt thereof, (b) a platelet aggregation inhibitor, and (c) a pharmaceutically acceptable carrier.

In a second aspect, the present invention provides a method of inhibiting platelet aggregation in a mammal comprising administering a therapeutically effective dose of: (a) a compound selected from pyridoxal-5′-phosphate, a pyridoxal-5′-phosphate related compound, or a pharmaceutically acceptable salt thereof, and (b) a platelet aggregation inhibitor.

In a third aspect, the present invention provides a method of treating a mammalian patient at risk for cardiovascular disease comprising administering a therapeutically effective dose of: (a) a compound selected from pyridoxal-5′-phosphate, a pyridoxal-5′-phosphate related compound, or a pharmaceutically acceptable salt thereof, and (b) a platelet aggregation inhibitor.

In an embodiment of the invention, the cardiovascular disease is congestive heart failure, myocardial ischemia, arrhythmia, myocardial infarction, ischemic stroke, hemorrhagic stroke, coronary artery disease, peripheral arterial disease, hypertension (high blood pressure), atherosclerosis (clogging of the arteries), aneurysm, thrombophlebitis (vein inflammation), diseases of the heart lining, diseases of the heart muscle, carditis, congestive heart failure, endocarditis, ischemic heart disease, valvular heart disease (malfunction of a valve or valves in the blood vessels of the heart), Kawazaki disease, ischemic injury, arteriosclerosis (hardening of the arteries), deep vein thrombosis, or acute coronary syndrome.

In a fourth aspect, the present invention provides a method of treating a mammalian patient at risk for cerebrovascular disease comprising administering a therapeutically effective dose of: (a) a compound selected from pyridoxal-5′-phosphate, a pyridoxal-5′-phosphate related compound, or a pharmaceutically acceptable salt thereof, and (b) a platelet aggregation inhibitor.

In an embodiment of the invention, the cerebrovascular disease is cerebral ischemia, cerebral hemorrhage, ischemic stroke, and hemmorrhagic stroke.

In a fifth aspect, the present invention provides a method of treating a mammal having a disease which arises from prothrombotic and thrombotic states in which the coagulation cascade is activated, comprising administering a therapeutically effective dose of: (a) a compound selected from pyridoxal-5′-phosphate, a pyridoxal-5′-phosphate related compound, or a pharmaceutically acceptable salt thereof, and (b) a platelet aggregation inhibitor

In an embodiment of the invention, the disease arising from prothrombotic and thrombotic states in which the coagulation cascade is activated is deep vein thrombosis, disseminated intravascular coagulopathy, or pulmonary embolism.

In a sixth aspect, the present invention provides a method for treating a mammalian patient undergoing a cardiovascular surgical intervention comprising administering a therapeutically effective dose of (a) a compound selected from pyridoxal-5′-phosphate, a pyridoxal-5′-phosphate related compound, or a pharmaceutically acceptable salt thereof, and (b) a platelet aggregation inhibitor, prior to the surgical intervention or following the surgical intervention.

In an embodiment of the invention, the surgical intervention is a coronary artery bypass graft (CABG), a percutaneous coronary intervention, or placement of a coronary stent.

In a seventh aspect, the present invention provides a use of: (a) a compound selected from pyridoxal-5′-phosphate, a pyridoxal-5′-phosphate related compound, or a pharmaceutically acceptable salt thereof, and (b) a platelet aggregation inhibitor, for the preparation of a medicament.

In an eighth aspect, the present invention provides a use of: (a) a compound selected from pyridoxal-5′-phosphate, a pyridoxal-5′-phosphate related compound, or a pharmaceutically acceptable salt thereof, and (b) a platelet aggregation inhibitor, for inhibiting platelet aggregation.

In a ninth aspect, the present invention provides a use of: (a) a compound selected from pyridoxal-5′-phosphate, a pyridoxal-5′-phosphate related compound, or a pharmaceutically acceptable salt thereof, and (b) a platelet aggregation inhibitor, for reducing the risk of a condition selected from a group consisting of: cardiovascular disease, cerebrovascular disease, and a disease which arises from prothrombotic and thrombotic states in which the coagulation cascade is activated.

In a tenth aspect, the present invention provides a use of: (a) a compound selected from pyridoxal-5′-phosphate, a pyridoxal-5′-phosphate related compound, or a pharmaceutically acceptable salt thereof, and (b) a platelet aggregation inhibitor, for treatment and prevention of thrombosis following a surgical intervention.

In a further embodiment of the invention, the pyridoxal-5′-phosphate related compound is pyridoxal, pyridoxal-5′-phosphate, pyridoxamine, a 3-acylated analogue of pyridoxal, a 3-acylated analogue of pyridoxal-4,5-aminal, a pyridoxine phosphate analogue, or a mixture thereof.

In another embodiment of the invention, the platelet aggregation inhibitor is a thromboxane A₂ inhibitor, a glycoprotein IIb/IIIa inhibitor, an adenosine diphosphate antagonist, a fibrinogen-platelet binding inhibitor, or a cAMP phosphodiesterase inhibitor.

### BRIEF DESCRIPTION OF THE DRAWINGS

FIG. 1 comprises line graphs illustrating the area under the curve CK-MB values fitted to a log-normal distribution for patients treated with P5P (A) and placebo (B).

### DETAILED DESCRIPTION

It is to be understood that this invention is not limited to specific dosage forms, carriers, or the like, and as such may vary. It is also to be understood that the terminology used herein is for the purpose of describing particular embodiments only, and is not intended to be limiting.

As used in this specification and the appended claims, the singular forms “a,” “an”, and “the” include plural referents unless the context clearly dictates otherwise. Thus, for example, reference to “an active agent” or “a pharmacologically active agent” includes a single active agent as well as two or more different active agents in combination, reference to “a carrier” includes mixtures of two or more carriers as well as a single carrier, and the like.

The term “pharmaceutically acceptable,” such as in the recitation of a “pharmaceutically acceptable carrier,” or a “pharmaceutically acceptable salt,” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained.

The terms “Carriers” and “vehicles” refer to conventional pharmaceutically acceptable carrier materials suitable for drug administration, and include any such materials known in the art that are nontoxic and do not interact with other components of a pharmaceutical composition or drug delivery system in a deleterious manner.

The terms “effective” amount or a “therapeutically effective amount” of a drug or pharmacologically active agent refers to a nontoxic but sufficient amount of the drug or agent to provide the desired effect. In the combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The terms “reduce the risk of cardiovascular disease” and “reducing the risk of cardiovascular disease” refer to the reduction or elimination of an underlying cause or biomarker associated with the increased incidence of a cardiovascular event.

As used herein, the term “cardiovascular disease” refers to any disease of the heart or blood vessels. Examples of cardiovascular disease include, but are not limited to: congestive heart failure, myocardial ischemia, arrhythmia, myocardial infarction (MI), ischemic stroke, hemorrhagic stroke, coronary artery disease, hypertension (high blood pressure), atherosclerosis (clogging of the arteries), aneurysm, peripheral artery disease (PAD), thrombophlebitis (vein inflammation), diseases of the heart lining, diseases of the heart muscle, carditis, congestive heart failure, endocarditis, ischemic heart disease, valvular heart disease (malfunction of a valve or valves in the blood vessels of the heart), arteriosclerosis (hardening of the arteries), acute coronary syndrome (ACS), high cholesterol, deep vein thrombosis (DVT), Kawazaki disease, peripheral vascular disease, ischemic injury, and heart transplant.

As used herein, the term “cerebrovascular disease” refers to any disease affecting blood supply to the brain. Examples of cerebrovascular disease include, but are not limited to: cerebral ischemia, cerebral hemorrhage, ischemic stroke, or hemorrhagic stroke.

As used herein, “a disease which arises from prothrombotic and thrombotic states in which the coagulation cascade activated” or a state of hypercoagulability, refers to any disease inherited or acquired or both, that meets the requirements of having one or more of Virchow's triad: a) changes in the vessel wall, b) changes in the pattern of blood flow, and c) changes in the constituents of blood, and is associated with a predisposition to venous thrombosis and/or arterial thrombosis. For the inherited diseases, common risk factors include, but are not limited to; antithrombin deficiencies, Protein C deficiencies, Protein S deficiencies, Factor V Leiden deficiencies, Dysfibrinogenemia Factor XII deficiencies, prothrombin 20210 mutations, hyperhomocystinemia, elevated factor XIII levels, and disorders of plasmin generation. For acquired hypercoagulable conditions, risk factors include, but are not limited to; pregnancy, immobility, trauma, postoperative state, use of oral contraceptives, use of estrogen and antiphospholipid syndrome. Examples of such diseases include, but are not limited to: deep vein thrombosis, disseminated intravascular coagulopathy, and pulmonary embolism.

As used herein, the terms “pyridoxal-5′-phosphate compound” or “pyridoxal-5′-phosphate related compound” refer to any vitamin B6 precursor, metabolite, derivative, or analogue but excludes vitamin B6 (pyroxidine).

As used herein, the terms “platelet aggregation inhibitor” and “antiplatelet agent” refer to any compound which inhibits activation, aggregation, and adhesion of platelets

The antithrombotic effect of vitamin B6 is known in the art. The present inventors have discovered that the platelet aggregation inhibition properties of pyridoxal-5′-phosphate and pyridoxal-5′-phosphate related compounds are significantly greater than those for vitamin B6 (pyroxidine). The present inventors have now discovered that pyridoxal-5′-phosphate and/or pyridoxal-5′-phosphate related compounds in combination with presently available platelet aggregation inhibitors, reduce the formation of blood clots in a enhanced manner and are effective for reducing the risk of cardiovascular disease and lowering the incidence of a cardiovascular event.

In view of these discoveries, the present invention provides novel pharmaceutical compositions and uses thereof for inhibiting platelet aggregation, treating disease which arises from prothrombotic and thrombotic states in which the coagulation cascade is activated and reducing the risk of cardiovascular disease. The pharmaceutical compositions of the present invention are more effective than currently available combination antiplatelet therapies. Furthermore, the pharmaceutical compositions ameliorate multiple risk factors for cardiovascular disease including lipoproteins, homocysteine, vasoconstriction, and inflammation. The pharmaceutical compositions of the present invention are comprised of a platelet aggregation inhibitor, a pyridoxal-5′-phosphate or pyridoxal-5′-phosphate related compound or a pharmaceutically acceptable salt thereof, and a pharmaceutically acceptable carrier.

Examples of known platelet aggregation inhibitors that may be used in accordance with the present invention include, but are not limited to: thromboxane A₂ inhibitors (e.g. acetylsalicylic acid (ASA)), glycoprotein IIb/IIIa inhibitors (e.g. abciximab, eptifibatide, tirofiban, lamifiban, xemilofiban, orbofiban, sibrafiban, fradafiban, roxifiban, lotrafiban), adenosine diphosphate (ADP) antagonist (e.g. clopidogrel (Plavix®), ticlopidine, sulfinpyrazone, AZD6140, AZD6933), cAMP phosphodiesterase inhibitors (e.g. dipyridamole, cilostazol (Pletal®), pentoxifylline (Trental®)) or fibrinogen-platelet binding inhibitors (e.g. ticlopidine).

The pharmaceutical compositions according to the invention can be prepared with a compound selected from: pyridoxal-5′-phosphate, a pharmaceutically acceptable salt of pyridoxal-5′-phosphate, a pyridoxal-5′-phosphate related compound, or a pharmaceutically acceptable salt of a pyridoxal-5′-phosphate related compound. Preferably, the pharmaceutical compositions according to the invention comprise pyridoxal-5′-phosphate.

Examples of pyridoxal-5′-phosphate related compounds that may be used in accordance with the present invention include, but are not limited to: pyridoxal-5-phosphate (P5P), pyridoxal, and pyridoxamine. Other pyridoxal-5′-phosphate related compounds which can also be used, including the 3-acylated analogues of pyridoxal, 3′acylated analogues of pyridoxal-4,5-aminal, and pyridoxine phosphonate analogues as disclosed in U.S. Pat. Nos. 6,339,085; 6,605,612; 6,667,315; 6,861,439; and 6,890,943; and U.S. Patent Application Publication Nos. 2003/0114677 and 2003/0195236, which are all hereby incorporated by reference.

The 3-acylated analogue of pyridoxal includes: 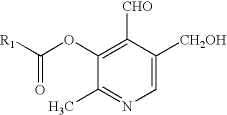
wherein,
R₁ is
   alkyl,
   alkenyl,
      in which alkyl or alkenyl
         can be interrupted by nitrogen, oxygen, or sulfur, and
         can be substituted at the terminal carbon by hydroxy, alkoxy, alkanoyloxy, alkanoyloxyaryl, alkoxyalkanoyl, alkoxycarbonyl, or dialkylcarbamoyloxy;
   alkoxy;
   dialkylamino;
   alkanoyloxy;
   alkanoyloxyaryl;
   alkoxyalkanoyl;
   alkoxycarbonyl;
   dialkylcarbamoyloxy; or
   aryl, in which aryl can be substituted by alkyl, alkoxy, amino, hydroxy, halo, nitro, or alkanoyloxy
   aryloxy,
   arylthio, or
   aralkyl, or a pharmaceutically acceptable acid addition salt thereof.

The 3-acylated analogue of pyridoxal-4,5-aminal includes: 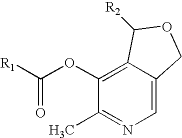
wherein,
R₁ is
   alkyl,
   alkenyl,
      in which alkyl or alkenyl
         can be interrupted by nitrogen, oxygen, or sulfur, and
         can be substituted at the terminal carbon by hydroxy, alkoxy, alkanoyloxy, alkanoyloxyaryl, alkoxyalkanoyl, alkoxycarbonyl, or dialkylcarbamoyloxy;
   alkoxy;
   dialkylamino;
   alkanoyloxy;
   alkanoyloxyaryl;
   alkoxyalkanoyl;
   alkoxycarbonyl;
   dialkylcarbamoyloxy; or
   aryl, in which aryl can be substituted by alkyl, alkoxy, amino, hydroxy, halo, nitro, or alkanoyloxy
   aryloxy,
   arylthio, or
   aralkyl; and
   R₂ is a secondary amino group, or a pharmaceutically accpetable acid addition salt thereof.

The pyridoxine phosphate analogue includes:
(a) 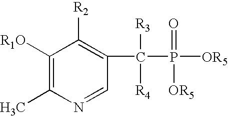
wherein,
R₁ is hydrogen or alkyl;
   R₂ is —CH₂OH, —CH₃, —CO₂R₆ in which R₆ is hydrogen, alkyl, or aryl; or
   R₂ is —CH₂—O-alkyl- in which alkyl is covalently bonded to the oxygen at the 3-position instead of R₁;
   R₃ is hydrogen and R₄ is hydroxy, halo, alkoxy, alkylcarbonyloxy, alkylamino or arylamino; or
   R₃ and R₄ are halo; and
   R₅ is hydrogen, alkyl, aryl, aralkyl, or —CO₂R₇ in which R₇ is hydrogen, alkyl, aryl, or aralkyl;
      or a pharmaceutically acceptable acid addition salt thereof;
      (b) 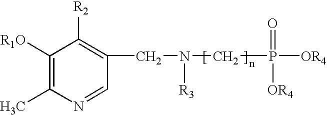
      wherein,
R₁ is hydrogen or alkyl;
   R₂ is —CHO, —CH₂OH, —CH₃ or —CO₂R₅ in which R₅ is hydrogen, alkyl, or aryl; or
   R₂ is —CH₂—O-alkyl- (in which alkyl is covalently bonded to the oxygen at the 3-position instead of R₁);
   R₃ is hydrogen, alkyl, aryl, or aralkyl;
   R₄ is hydrogen, alkyl, aryl, aralkyl, or —CO₂R₆ in which R₆ is hydrogen, alkyl, aryl, or aralkyl; and
   n is 1 to 6;
      or a pharmaceutically acceptable acid addition salt thereof; and
      (c) 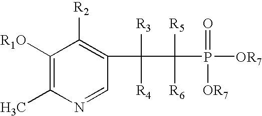
      wherein,
R₁ is hydrogen or alkyl;
   R₂ is —CHO, —CH₂OH, —CH₃ or —CO₂R₈ in which R₈ is hydrogen, alkyl, or aryl; or
   R₂ is —CH₂—O-alkyl- in which alkyl is covalently bonded to the oxygen at the 3-position instead of R₁;
   R₃ is hydrogen and R₄ is hydroxy, halo, alkoxy or alkylcarbonyloxy; or
   R₃ and R₄ can be taken together to form ═O;
   R₅ and R₆ are hydrogen; or
   R₅ and R₆ are halo; and
   R₇ is hydrogen, alkyl, aryl, aralkyl, or —CO₂R₈ in which R₈ is hydrogen, alkyl, aryl, or aralkyl;
   or a pharmaceutically acceptable acid addition salt thereof.

As used herein “alkyl” includes a saturated linear or branched hydrocarbon radical. In one embodiment, alkyl has from 1 to 8 carbon atoms. In another embodiment, alkyl has from 1 to 6 carbon atoms. In another embodiment, alkyl has from 1 to 4 carbon atoms. In one embodiment, alkyl has 1 carbon. The alkyl group may optionally be substituted with one or more substituents such as fluorine, chlorine, alkoxy groups having from 1 to 8 carbon atoms (e.g., methoxy or ethoxy), or amido groups having from 1 to 8 carbon atoms, such as acetamido. These substituents may themselves be substituted with one or more functional groups such as hydroxy groups, carboxy groups, acetoxy groups, or halogens.

As used herein “aryl” means a mono- or poly-nuclear aromatic hydrocarbon radical. Examples of “aryl” groups include, but are not limited to aromatic hydrocarbons such as a phenyl group or a naphthyl group. The aromatic group may optionally be substituted with one or more substituents such as fluorine, chlorine, alkyl groups having from 1 to 8 carbon atoms (e.g., methyl or ethyl), alkoxy groups having from 1 to 8 carbon atoms (e.g., methoxy or ethoxy), alkoxyalkyl groups having from 1 to 8 carbon atoms and one or more oxygen atoms, or amido groups having from 1 to 8 carbon atoms, such as acetamido. These substituents may themselves be substituted with one or more functional groups such as hydroxy groups, carboxy groups, acetoxy groups, or halogens.

In one embodiment, aryl is a phenyl group or a naphthyl group that is either unsubstituted or substituted.

In another embodiment, aryl is a heteroaryl in which one or more of the carbon atoms of an aromatic hydrocarbon is substituted with a nitrogen, sulfur, or oxygen. Examples of a “heteroaryl” include, but are not limited to pyridine, pyrimidine, pyran, dioxin, oxazine, and oxathiazine. Likewise, the heteroaryl may optionally be substituted with functional groups such as hydroxy groups, carboxy groups, halogens, and amino groups.

The term “alkenyl” includes an unsaturated aliphatic hydrocarbon chain having from 2 to 8 carbon atoms, such as, for example, ethenyl, 1-propenyl, 2-propenyl, 1-butenyl, 2-methyl-1-propenyl, and the like.

The above alkyl or alkenyl can optionally be interrupted in the chain by a heteroatom, such as, for example, a nitrogen, sulfur, or oxygen atom, forming an alkylaminoalkyl, alkylthioalkyl, or alkoxyalkyl, for example, methylaminoethyl, ethylthiopropyl, methoxymethyl, and the like.

The above alkyl or alkenyl can optionally be substituted at the terminal carbon by hydroxy, alkoxy, alkanoyloxyaryl, alkanoyloxy, alkoxyalkanoyl, alkoxycarbonyl, or dialkylcarbamoyloxy.

The term “alkoxy” (i.e. alkyl-O—) includes alkyl as defined above joined to an oxygen atom having preferably from 1 to 4 carbon atoms in a straight or branched chain, such as, for example, methoxy, ethoxy, propoxy, isopropoxy (1-methylethoxy), butoxy, tert-butoxy (1,1-dimethylethoxy), and the like.

The term “dialkylamino” includes two alkyl groups as defined above joined to a nitrogen atom, in which alkyl has preferably 1 to 4 carbon atoms, such as, for example, dimethylamino, diethylamino, methylethylamino, methylpropylamino, diethylamino, and the like.

The term “alkanoyloxy” includes a group of the formula 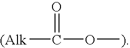
Examples of alkanoyloxy include methanoyloxy, ethanoyloxy, propanoyloxy, and the like. Examples of alkyl substituted at the terminal carbon by alkanoyloxy include 1-ethanoyloxy-1-methylethyl, propanoyloxy-1-methylethyl, and the like.

The term “alkanoyloxyaryl” includes a group of the formula 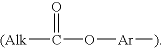
Examples of alkanoyloxyaryl include methanoyloxyphenyl, ethanoyloxyphenyl, propanoyloxyphenyl, and the like.

The term “aryl” refers to unsaturated aromatic carbocyclic radicals having a single ring, such as phenyl, or multiple condensed rings, such as naphthyl or anthryl. The term “aryl” also includes substituted aryl comprising aryl substituted on a ring by, for example, C₁₋₄ alkyl, C₁₋₄ alkoxy, amino, hydroxy, phenyl, nitro, halo, carboxyalkyl or alkanoyloxy. Aryl groups include, for example, phenyl, naphthyl, anthryl, biphenyl, methoxyphenyl, halophenyl, and the like.

The term “aryloxy” (i.e. aryl-O—) includes aryl having an oxygen atom bonded to an aromatic ring, such as, for example, phenoxy and naphthoxy.

The term “arylthio” (i.e. aryl-S—) includes aryl having a sulfur atom bonded to an aromatic ring, such as, for example, phenylthio and naphthylthio.

The term “aralkyl” refers to an aryl radical defined as above substituted with an alkyl radical as defined above (e.g. aryl-alkyl-). Aralkyl groups include, for example, phenethyl, benzyl, and naphthylmethyl.

Aryl from any of aryl, aryloxy, arylthio, aralkyl, and alkanoyloxyaryl can be unsubstituted or can be substituted on a ring by, for example, C₁₋₄ alkyl, C₁₋₄ alkoxy, amino, hydroxy, nitro, halo, or alkanoyloxy. Examples of substituted aryl include toluyl, methoxyphenyl, ethylphenyl, and the like.

The term “alkoxyalkanoyl” includes a group of the formula 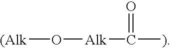
Examples of alkoxyalkanoyl include (2-acetoxy-2-methyl)propanyl, 3-ethoxy-3-propanoyl, 3-methoxy-2-propanoyl, and the like.

The term “alkoxycarbonyl” includes a group of the formula 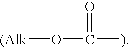
Examples of alkoxycarbonyl include methoxycarbonyl, ethoxycarbonyl, propoxycarbonyl, and the like.

The term “dialkylcarbamoyloxy” includes a group of the formula 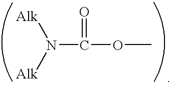
Examples of dialkylcarbamoyloxy include dimethylamino-methanoyloxy, 1-ethyl-1-methylaminomethanoyloxy, and the like. Examples of alkyl substituted at the terminal carbon by alkanoyloxy include dimethylamino-1-methylethyl, 1-ethyl-1-methylaminomethanoyloxy-1-methlethyl, and the like.

The term “halo” includes bromo, chloro, and fluoro.

The invention also includes pharmaceutically acceptable salts of the compounds of the invention. The compounds of the invention are capable of forming both pharmaceutically acceptable acid addition and/or base salts. Pharmaceutically acceptable acid addition salts of the compounds of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and di-carboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glucamine, etc. (see Berge et al., J. Pharmaceutical Science, 66: 1-19 (1977). The term “pharmaceutically acceptable salts” also includes any pharmaceutically acceptable base salt including, but not limited to, amine salts, trialkyl amine salts and the like. Such salts can be formed quite readily by those skilled in the art using standard techniques.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention. Base salts are formed with metals or amines, such as alkali and alkaline earth metals or organic amines. Examples of metals used as cations include, but are not limited to, sodium, potassium, magnesium, and calcium. Examples of suitable amines are N,N′-dibenzylethylenediamine, chloroprocaine, choline, diethanolamine, ethylenediamine, N-methylglucamine, and procaine.

Some of the compounds described herein contain one or more asymmetric centers and may thus give rise to enantiomers, diastereomers, and other stereoisomeric forms which may be defined in terms of absolute stereochemistry as (R)- or (S)-. The present invention is meant to include all such possible diastereomers and enantiomers as well as their racemic and optically pure forms. Optically active (R)- and (S)-isomers may be prepared using chiral synthons or chiral reagents, or resolved using conventional techniques. When the compounds described herein contain centers of geometric asymmetry, and unless specified otherwise, it is intended that the compounds include both E and Z geometric isomers. Likewise all tautomeric forms are intended to be included.

The pharmaceutical composition according to the invention may be prepared using pyridoxal 5′-phosphate, a pharmaceutically acceptable salt of pyridoxal 5′-phosphate, a pyridoxal 5′-phosphate related compound, or a pharmaceutically acceptable salt of a pyridoxal 5′-phosphate related compound. Preferably, pharmaceutical compositions are prepared using pyridoxal 5′-phosphate. Both the monohydrate and the anhydrous forms of pyridoxal 5′-phosphate are suitable for preparation of the pharmaceutical compositions of the invention. Pyridoxal 5′-phosphate or the pyridoxal 5′-phosphate related compound may be provided as salt forms with pharmaceutically compatible counterions such as but not limited, to citrate, tartate, bisulfate, etc. The pharmaceutically compatible salts may be formed with many acids, including but, not limited to, hydrochloric, sulfuric, acetic, lactic, tartaric, malic, succinic, etc. The salt forms tend to be more soluble in aqueous or other protonic solvents than the corresponding free base forms.

In a preferred embodiment of the invention, the pharmaceutical composition comprises ASA and pyridoxal-5′-phosphate. In another preferred embodiment of the invention, the pharmaceutical composition comprises clopidogrel (Plavix®) and pyridoxal-5′-phosphate. In a further preferred embodiment of the invention, the pharmaceutical composition comprises eptifibatide (Integrilin®) and pyridoxal-5′-phosphate.

Pharmaceutical compositions for use in accordance with the present invention may be formulated in a conventional manner using one or more physiologically acceptable carriers comprising excipients and auxiliaries that facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or a physiological saline buffer.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol, or cellulose preparations such as, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents may be added, such as the cross-linked polyvinylpyrrolidone, agar, or alginic acid or a salt thereof such as sodium alginate.

Preferably, the pharmaceutical compositions of the present invention are administered orally. Preferred oral dosage forms contain a therapeutically effective unit dose of each active agent, wherein the unit dose is suitable for a once-daily oral administration. The therapeutic effective unit dose of any of the active agents will depend on number of well known factors. In particular these factors include: the identity of the compounds to be administered, the formulation, the route of administration employed, the patient's gender, age, and weight, and the severity of the condition being treated. Where the dose provided does not reduce platelet aggregation levels, as measured by the closure time (CL) using, for example the Platelet Function Analyzer PFA-100®, or by measuring the bleeding time (BL), to appropriate levels, following at least 10 days of treatment, the dose can be increased.

The therapeutic effective unit dosage for the platelet aggregation inhibitor will vary depending on the particular inhibitor used and the condition to be treated. The pharmaceutical compositions according to the invention can be used in cases where it is desirable to inhibit platelet aggregation. The pharmaceutical compositions according to the invention can also be used to treat patients at risk of a cardiovascular disease. The pharmaceutical compositions according to the invention can further be used to treat a patient undergoing a surgical intervention and preferably a cardiovascular surgical intervention such as but not limited to: a coronary artery bypass graft, a percutaneous coronary intervention or placement of a coronary stent. The pharmaceutical compositions can be used to treat or prevent the occurrence of thrombosis following the surgical intervention.

Where the platelet aggregation inhibitor used is ASA and it is used for the prevention of myocardial infarction (MI), transient ischemic attack (TIA), or ischemic stroke, the therapeutic effective unit dosage can be between 5 to 500 mg per day, and preferably between 30 mg and 81 mg per day. More preferably, the unit dosage will be between 75 mg and 81 mg per day and even more preferably, the unit dosage will be 81 mg per day. When ASA is used postoperatively in the case of a coronary artery bypass graft (CABG) or a percutaneous coronary intervention (PCI), the effective dose is preferably 325 mg three times daily, continued until further notice from a physician.

Where the platelet aggregation inhibitor used is eptifibatide and it is used for prophylaxis of percutaneous coronary intervention (PCI) related thrombosis, the therapeutic effective unit dosage is between 30 to 500 μg/kg. A bolus IV injection of 135 μg/kg can be administered immediately before surgery and a continuous IV infusion of between 0.1 to 5 μg/kg/min and more preferably a continuous IV infusion of 0.5 μg/kg/min, can be administered 20 to 24 hours after surgery.

When the platelet aggregation inhibitor eptifibatide used for the treatment of acute coronary syndrome, the therapeutic effective unit dosage of eptifibatide is preferably between 30 to 500 μg/kg. A bolus injection of 180 μg/kg can be administered as soon as possible after diagnosis, immediately followed by continuous IV infusion of between 0.1 to 5 μg/kg/min, and more preferably a continuous IV infusion of 2 μg/kg/min until hospital discharge (up to 72 hours).

When the platelet aggregation inhibitor eptifibatide is used for prophylaxis for coronary stenting, the therapeutic effective unit dosage of eptifibatide is preferably between 30 to 500 μg/kg. Preferably, the eptifibatide can be administered as a first bolus injection of 180 μg/kg followed by a continuous infusion of between 0.1 to 5 μg/kg/min, and more preferably, a continuous IV infusion of 2 μg/kg/min for 10 minutes, which is then followed by a second bolus injection of 180 μg/kg. A continuous infusion can then be resumed for 18 to 24 hours.

When the platelet aggregation inhibitor clopidogrel is used as a prophylaxis for MI, stroke, or thrombotic or vascular injury, the therapeutic effective unit dosage is between 10 and 1000 mg per day and preferably between 75 mg and 150 mg per day. More preferably the unit dosage per day would be 75 mg. When used immediately before surgery, the therapeutic effective dosage unit would be between 300 mg and 500 mg. More preferably, the unit dosage would be 300 to 350 mg and even more preferably the unit dosage would be 300 mg.

The preferable therapeutic effective unit dosage for the pyridoxal-5′-phosphate or pyridoxal-5′-phosphate related compound is between 0.1 to 50 mg/kg body weight daily. More preferably, the unit dosage will be 1 to 5 mg/kg body weight daily.

For reducing the risk of cerebrovascular disease, a similar dose range of 0.1-100 mg/kg or more preferably 0.5 to 50 mg orally, can be used. For pyridoxal-5′-phosphate, the dosage used would be similar, e.g. 1 mg/kg to 15 mg/kg per day given intravenously to the patient immediately after the stroke, until otherwise directed by physician. More preferably, the dosage will be 10 to 15 mg/kg per day given intravenously. For reducing the risk of cardiovascular disease, the daily dosage may be the same as for stroke.

Although the present invention has been described with reference to illustrative embodiments, it is to be understood that the invention is not limited to these precise embodiments, and that various changes and modifications may be effected therein by one skilled in the art. All such changes and modifications are intended to be encompassed in the appended claims.

### EXAMPLE 1

### Effectiveness of pyridoxal-5′-phosphate for the Reduction of Myocardial Ischemic Injury Following Coronary Intervention

Methods—Study Overview: 60 patients who underwent percutaneous coronary intervention (PCI) at 4 centers were randomized in a 2:1 double-blinded fashion to treatment with P5P or placebo. Inclusion criteria required prior determination for non-urgent PCI of a single-vessel lesion(s) and identification of ≧1 of the following clinical characteristics determining high risk for procedural-related ischemic complications (Califf R M, Abdelmeguid A E, Kuntz R E, Popma J J, Davidson C J, Cohen E A, Kleiman N S, Mahaffey K W, Topol E J, Pepine C J, et al. Myonecrosis after revascularization procedures. J Am Coll Cardiol 1998; 31:241-251; The ESPRIT Investigators. Novel dosing regimen of eptifibatide in planned coronary stent implantation: a randomised, placebo-controlled trial. Lancet 2000; 356:2037-2044): presence of an acute coronary syndrome (chest pain within 48 hours of PCI), recent AMI (≦7 days), diminished epicardial blood flow, angiographic thrombus, ejection fraction ≦30%, or vein graft lesion. In addition to any general contraindication to the PCI procedure or standard concomitant therapies, major exclusion criteria were creatine kinase (CK-MB) elevation above the upper limit of normal immediately before PCI, electrocardiographic evidence of atrial fibrillation or left bundle branch block, or evidence of any clinically significant abnormal laboratory finding (transaminases, bilirubin, or alkaline phosphatase >1.5 times the upper limit of normal or serum creatinine >1.8 mg/dl). Patients with elevated troponin measurements were permitted in the study provided that the peak troponin value was reported >24 hours before scheduled PCI, with documentation of a decreasing value before revascularization. After providing informed consent, patients randomized to treatment with P5P were administered enteric-coated P5P as a 10 mg/kg oral dose ≧4 hours before PCI followed by 2 daily doses of 5 mg/kg orally for 14 days. Compliance and reasons for discontinued treatments were recorded for all patients.

Study end points and definitions: The primary objective of the study was to evaluate the feasibility of treatment with P5P as a cardioprotective agent in high-risk elective PCI. The primary end point of infarct size was evaluated by the trapezoidal rule (Press W H, Teukolsky S A, Vetterling W T, Flannery B P. Numerical Recipes. Cambridge, UK: Cambridge University Press, 1994:127-133) using serial CK-MB enzyme measures performed at baseline and every 6 hours for 24 hours beginning immediately before initiation of PCI. The occurrence of myocardial ischemia within 24 hours after PCI was assessed as a secondary end point using continuous 12-lead electrocardiographic monitoring (Northeast Monitoring, Boston, Mass.). Evidence of periprocedural ischemia was defined as ST-segment depression of >100 μV within a 60-minute period of PCI, lasting ≧1 minute and separated from other episodes by ≧1 minute. Area under the curve ST-segment deviation was measured from the onset of the first to the last contrast injection. All cardiac markers and ST-segment monitoring data were analyzed by core laboratories blinded to treatment assignment (University of Maryland School of Medicine, Baltimore, Md.; Duke Ischemia Monitoring Laboratory, Durham, N.C.). Additional prespecified secondary end points included the 30-day composite and individual event rates of death; nonfatal infarction; new or worsening heart failure, or recurrent ischemia in addition to net clinical safety, which was defined as the absence of major adverse ischemic events; thrombolysis in myocardial infarction (TIMI) major bleeding; and liver function or coagulation test abnormalities. Acute myocardial infarction (AMI) was defined as CK-MB elevation ≧3 times the upper limit of normal (upper limit of normal 7 ng/ml) and/or troponin T levels ≧1.5 times the upper limit of normal (upper limit of normal 0.1 ng/ml). If previous troponin (or CKMB) values were above the upper limit of normal, values were required to be >50% of the baseline measurement in addition to ≧2 times (≧3 times for CK-MB) the upper limit of normal to meet the definition of AMI. Routine chemistries, complete blood count, and coagulation assays were performed at baseline, 7 days, and 30 days after randomization. Peak periprocedural CK-MB and the maximum difference in troponin levels from baseline to within 24 hours after PCI were also examined.

Data collection and statistical analyses: Patients who received ≧1 dose of the study drug and underwent PCI were analyzed for all primary and secondary efficacy and safety end points. Patients who received ≧1 dose of study drug but who did not undergo PCI were excluded from the primary efficacy and ST segment monitoring analyses but were included in the safety analyses. Statistical tests were 2-sided with an a level of 0.05 and employed the intent-to-treat principle. The Wilcoxon rank-sum test was used to analyze all continuous variables. Due to small sample sizes, categorical variables were compared using the Fisher's exact test with the exception of the ST-segment monitoring data, which utilized the Pearson's chi-square test. Statistical analyses were performed using SAS version 8.2 (SAS Institute, Cary, N.C.).

Results—Of the 60 patients enrolled in the study of P5P in high-risk PCI, all patients received treatment with P5P or placebo; however, 4 patients (3 P5P, 1 placebo) did not undergo planned revascularization. An additional 3 patients were excluded from the area under the curve analyses due to incomplete collection of cardiac enzyme data. As a result, 53 and 60 patients were included in the primary efficacy and 30-day clinical and/or safety analyses, respectively.

The presence of established cardiovascular disease, prior revascularization, and cardiovascular risk factors were similar between patients randomized to P5P or placebo and representative of patient populations in larger contemporary trials that studied patients with acute coronary syndromes (Table 1). Overall, the mean age of the population was 58 years, 81.7% of patients were men, and 21.7% had undergone previous PCI and/or bypass surgery. Although recent AMI as an indication for revascularization occurred more commonly among patients treated with P5P, a similar number of patients in each group presented with an acute coronary syndrome, and approximately half of all patients had elevated troponin levels before PCI.

Except for a higher incidence of reduced epicardial flow among control patients, baseline angiographic and procedural characteristics also appeared similar between treatment groups (Table 1). Administration of P5P or placebo occurred an average of 6.1 and 8.4 hours before PCI, respectively. Stent implantation was performed in 100% and 97.3% of the placebo and P5P treatment groups, respectively. Only 1 vein graft intervention was performed using distal embolic protection. Although the right coronary artery was most commonly treated in both groups, fewer patients treated with placebo underwent revascularization of a saphenous vein graft (Table 2). Procedural angiographic complications (e.g., major dissection, abrupt vessel closure) were infrequent (Table 2).

TABLE 1
Baseline Clinical Electrocardiographic, and Angiographic Characteristics
in patients treated with pyridoxal-5′-phosphate (P5P) or placebo.

| | PSP (n = 40) | Placebo (n = 20) |
|---|---|---|
| Clinical Characteristics* | | |
| Age (yrs) (range) | 54 (48-66) | 59 (55-69) |
| Men | 32 (80) | 17 (85) |
| Baseline troponin positive | 14/30 (47) | 6/14 (43) |
| Diabetes mellitus | 9 (23) | 4 (20) |
| Systemic hypertension | 17 (43) | 9 (45) |
| Hyperlipidemia (requiring medical | 31(78) | 17 (85) |
| treatment or LDL> 130 mg/dl) | | |
| Current smoker | 12 (30) | 5 (25) |
| Prior myocardial infarction | 14 (35) | 9 (45) |
| Prior PCI | 6 (15) | 2 (10) |
| Prior coronary bypass graft | 5 (13) | 2 (10) |
| surgery | | |
| Prior stroke or transient ischemic | 1 (3) | 1 (5) |
| attack | | |
| Peripheral vascular disease | 3 (8) | 7 (35) |
| Congestive heart failure | 3 (8) | 2 (10) |
| Qualifying electrocardiogram | | |
| ST-segment depression | 2 (5) | 2 (10) |
| ST-segment elevation | 7 (18) | 2 (10) |
| T-wave inversion | 6 (15) | 4 (20) |
| Angiographic characteristics | | |
| PCI performed | 37 (93) | 19 (95) |
| Reason for PCI | (n = 37) | (n = 19) |
| Acute coronary syndrome | 9 (24) | 5 (25) |
| Recent AMI | 16 (42) | 3 (15) |
| Reduced epicardial flow | 6 (16) | 8 (40) |
| Thrombus | 1 (3) | 1 (5) |
| Congestive heart failure | 2 (5) | 1 (5) |
| Saphenous vein graft lesion | 4 (11) | 2 (10) |
| No. of coronary arteries narrowed | (n = 37) | (n = 19) |
| ≧50% | | |
| In diameter | | |
| 0 | 1 (3) | 0 |
| 1 | 19 (48) | 14 (70) |
| 2 | 13 (33) | 2 (10) |
| 3 | 5 (13) | 3 (15) |
| Left main | 2 (5) | 1 (5) |
| Left ventricular ejection fraction | 0.50 (0.40-0.68) | 0.56 (0.37-0.64) |
| No. of coronary narrowings treated | (n = 37) | (n = 19) |
| 1 | 26 (70) | 15 (79) |
| 2 | 8 (22) | 3 (16) |
| 3 | 3 (8) | 1 (5) |

Values are expressed as median (Interquartile range) or number (percent).
*Patients may be double counted
IDL = low-density lipoprotein

TABLE 2
Procedural and Angiographic* results in patients treated with P5P or
placebo.

| | P5P (n = 37) | Placebo (n = 19) |
|---|---|---|
| ≧1 stent implanted | 36 (97) | 19 (100) |
| Patients received GP llb/llla | 29/35 (83) | 15/19 (79) |
| inhibitor | | |
| Target vessel | | |
| Left anterior descending | 11 (30) | 4 (21) |
| Right | 14 (38) | 11 (58) |
| Left cirumfiex | 8 (22) | 3 (16) |
| Saphenous vein graft | 4 (11) | 1 (5) |
| TIMI flow prepocedure | | |
| 0/1 | 3 (8) | 4 (22) |
| 2 | 7 (19) | 4 (22) |
| 3 | 27 (73) | 10 (56) |
| TIMI flow final | | |
| 0/1 | 0 | 0 |
| 2 | 0 | 1 (5) |
| 3 | 37 (100) | 18 (95) |
| Diameter stenosis preprocedure | 90.0 (80.0-95.0) | 95.0 (90.0-99.0) |
| % | | |
| Diameter stenosis final (%) | 0 (0-0) | 0 (0-0) |
| Procedural complications | | |
| None | 35 (95) | 18 (95) |
| Major dissection | 1 (3) | 1 (5) |
| Abrupt closure | 0 | 0 |
| No reflow | 0 | 0 |
| Thrombus formation | 0 | 0 |
| Side branch closure | 1 (3) | 0 |
| Distal embolization | 0 | 0 |

Values are expressed as median (lnterquartile range) or number (percent)
*Investigator-reported angiographic values
GP = glycoprotein

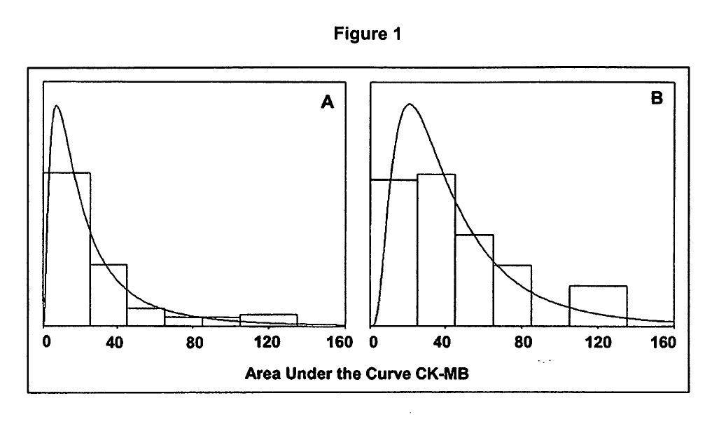

The primary end point of periprocedural infarct size measured according to median periprocedural CK-MB area under the curve was reduced from 32.9 to 18.6 ng/ml (p=0.038), reflecting a shift in the distribution of CK-MB (Table 3 and FIG. 1). Similarly, the maximum periprocedural CK-MB level was significantly lower among patients receiving P5P. By categorical classification, the occurrence of 30-day nonfatal AMI did not differ between groups (12.8% with P5P vs 10.0% with placebo, p=1.0). There were no deaths, and 30-day composite adverse event rates (death, nonfatal AMI, new and/or worsening heart failure, or recurrent ischemia) were similar (17.9% with P5P vs 15.0% with placebo, p=1.0).

Electrocardiographic ST monitoring data were available for 94.6% of the patients who underwent PCI and who received treatment (Table 3). Post-PCI ischemia occurred in approximately 15% of patients in both groups. Although lower rates of post-PCI ischemia were observed with P5P treatment (14.7% vs 17.6%, p=0.78), there were no significant differences in ischemia parameters per continuous electrocardiographic monitoring (Table 3).

TABLE 3
Periprocedural Cardiac Markers and ST Monitoring Results results for patients treated with P5P or placebo.

| | P5P | Placebo | p Value |
|---|---|---|---|
| Periprocedural cardiac markers | | | |
| Area under the curve cK-MB (ng/ml) | 18.6 (10.2-34.5), 35 | 32.9 (19.4-64.3) 18 | 0.04 |
| Peak OK-MB (ng/ml) | 1.1 (0.5-2.4), 39 | 2.0 (1.4-6.3), 19 | 0.03 |
| change in troponin T (ng/ml) | 0 (0-0.07), 36 | 0(0-0.10), 19 | 0.65 |
| Time to peak CK-MB (h) | 11.0 (0-18.0), 36 | 14.0 (12.0-18.0), 19 | 0.10 |
| 24-h continuous electrocardiographic ST monitoring | | | |
| Duration of monitoring (h) | 22.6 (20.4.23.9), 36 | 22.4 (20.6-24.0), 17 | — |
| Area under the curve ST deviation (μV-min) | 1349 (951-2.263), 35 | 1603 (1,049-1.945), 17 | 0.49 |
| Any post PCI ischemia (%) | 14.7-34 | 17.6-17 | 0.78 |

Values are expressed in median (interguartle range) or percent followed by n (number of observations

No safety issues related to treatment with P5P were identified. The occurrence of major bleeding (2.8% P5P vs 10.5% placebo, p=0.27) and need for blood product transfusion (2.5% P5P vs 10.0% placebo, p=0.26) was infrequent and did not significantly differ between groups. There were no apparent differences in abnormalities of routine chemistries or coagulation studies at 7 and 30 days. In both groups, however, approximately ¼ of patients discontinued drug therapy before completion of the prescribed 2 weeks (30.8% P5P vs 25.0% placebo, p=0.77). For patients taking P5P, but who did not undergo PCI (3 patients, 7.5%), the most common causes for early discontinuation were gastrointestinal intolerance followed by non-specific musculoskeletal pain.

In high-risk patients for periprocedural ischemic complications, treatment with P5P was associated with a decrease in myocardial injury, reflected by a reduction in the total amount of CK-MB released after PCI. P5P therapy was associated with a significant decrease in peak periprocedural CK-MB elevation, a shift in the distribution of CK-MB to lower levels (FIG. 1), and reduced periprocedural infarct size.

### EXAMPLE 2

### Effectiveness of pyridoxal-5′-phosphate in Combination with Aspirin for the Reduction of Myocardial Ischemic Injury Following Coronary Intervention

Method: The study data of Example 1 was examined. Of the 60 patients described in Example 1, 35 patients received adjunctive treatment with acetylsalicylic acid [82 mg (6 patients) and 325 mg (29 patients)] in addition to P5P treatment.

Results: In patients treated with P5P and ASA, the secondary end point of maximum periprocedural CK-MB levels was reduced from 3.41 ng/ml (placebo and ASA) to 2.09 ng/ml (P5P and ASA; Table 4).

TABLE 4
Periprocedural cardiac markers results for patients treated with P5P in
combination with acetylsalicylic acid, eptifibatide, or clopidogrel and
patients treated with placebo in combination with acetylsalicylic acid,
eptifibatide, or clopidogrel.

| | CK-MB max (mean) | |
|---|---|---|
| Combination Therapy | ng/ml | Sample size |
| ASA + placebo | 3.41 | 18 |
| ASA + P5P | 2.09 | 35 |
| Eptifibatide + placebo | 3.40 | 9 |
| Eptifibatide + P5P | 1.36 | 19 |
| Clopidogrel + placebo | 3.41 | 14 |
| Clopidogrel + P5P | 2.14 | 25 |

P5P and ASA combination therapy was associated with a significant decrease in peak periprocedural CK-MB elevation, and reduced periprocedural infarct size.

### EXAMPLE 3

### Effectiveness of pyridoxal-5′-phosphate in Combination with Eptifibatide (Integrilin) for the Reduction of Myocardial Ischemic Injury Following Coronary Intervention

Methods: The study data of Example 1 was examined. Of the 60 patients described in Example 1, 19 patients received adjunctive treatment with eptifibatide in addition to P5P treatment.

Results: In patients treated with P5P and eptifibatide, the secondary end point of maximum periprocedural CK-MB levels was reduced from 3.40 ng/ml (placebo and eptifibatide) to 1.36 ng/ml (P5P and eptifibatide),

P5P and eptifibatide combination therapy was associated with a significant decrease in peak periprocedural CK-MB elevation (Table 4), and reduced periprocedural infarct size.

### EXAMPLE 4

### Effectiveness of pyridoxal-5′-phosphate in Combination with Clopidogrel (Plavix) for the Reduction of Myocardial Ischemic Injury Following Coronary Intervention

Methods: The study data of Example 1 was examined. Of the 60 patients described in Example 1, 25 patients received adjunctive treatment with clopidogrel (75 mg, 16 patients and 300 mg, 9 patients) in addition to P5P treatment.

Results: In patients treated with P5P and clopidogrel, the secondary end point of maximum periprocedural CK-MB levels was reduced from 3.41 ng/ml (placebo and clopidogrel) and to 2.14 ng/ml (P5P and clopidrogel),

P5P and clopidogrel combination therapy was associated with a significant decrease in peak periproceduarl CK-MB elevation (Table 4), and reduced periprocedural infarct size.

What is claimed is:

1. A pharmaceutical composition comprising: (a) a compound selected from pyridoxal-5′-phosphate, a pyridoxal-5′-phosphate related compound, or a pharmaceutically acceptable salt thereof; (b) a platelet aggregation inhibitor; and (c) a pharmaceutically acceptable carrier.

2. The pharmaceutical composition according to claim 1, wherein the pyridoxal-5′-phosphate related compound is selected from a group comprising: pyridoxal, pyridoxal-5′-phosphate, pyridoxamine, a 3-acylated analogue of pyridoxal, a 3-acylated analogue of pyridoxal-4,5-aminal, a pyridoxine phosphate analogue, and a mixture thereof.

3. The pharmaceutical composition according to claim 1, wherein the compound is pyridoxal-5′-phosphate.

4. The pharmaceutical composition according to claim 2, wherein the 3-acylated analogue of pyridoxal is: 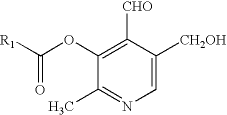
wherein,
R₁ is
alkyl,
alkenyl,
in which alkyl or alkenyl
can be interrupted by nitrogen, oxygen, or sulfur, and
can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy; or
aryl, in which aryl can be substituted by alkyl, alkoxy, amino, hydroxy, halo, nitro, or alkanoyloxy
aryloxy,
arylthio, or
aralkyl, or a pharmaceutically acceptable acid addition salt thereof.

5. The pharmaceutical composition according to claim 2, wherein the 3-acylated analogue of pyridoxal-4,5-aminal is 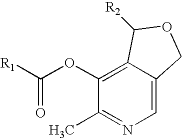
wherein,
R₁ is
alkyl,
alkenyl,
in which alkyl or alkenyl
can be interrupted by nitrogen, oxygen, or sulfur, and
can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy; or
aryl, in which aryl can be substituted by alkyl, alkoxy, amino, hydroxy, halo, nitro, or alkanoyloxy
aryloxy,
arylthio, or
aralkyl; and
R₂ is a secondary amino group, or a pharmaceutically accpetable acid addition salt thereof.

6. The pharmaceutical composition according to claim 2, wherein the pyridoxine phosphate analogue is selected from a group comprising:
(a) 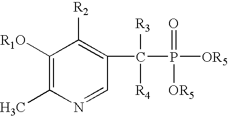
wherein,
R₁ is hydrogen or alkyl;
R₂ is —CH₂OH, —CH₃, —CO₂R₆ in which R₆ is hydrogen, alkyl, or aryl; or
R₂ is —CH₂—O-alkyl- in which alkyl is covalently bonded to the oxygen at the 3-position instead of R₁;
R₃ is hydrogen and R₄ is hydroxy, halo, alkoxy, alkylcarbonyloxy, alkylamino or arylamino; or
R₃ and R₄ are halo; and
R₅ is hydrogen, alkyl, aryl, aralkyl, or —CO₂R₇ in which R₇ is hydrogen, alkyl, aryl, or aralkyl;
or a pharmaceutically acceptable acid addition salt thereof;
(b) 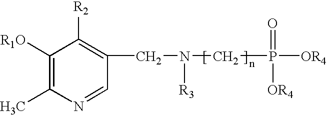
wherein,
R₁ is hydrogen or alkyl;
R₂ is —CHO, —CH₂OH, —CH₃ or —CO₂R₅ in which R₅ is hydrogen, alkyl, or aryl; or
R₂ is —CH₂—O-alkyl- (in which alkyl is covalently bonded to the oxygen at the 3-position instead of R₁);
R₃ is hydrogen, alkyl, aryl, or aralkyl;
R₄ is hydrogen, alkyl, aryl, aralkyl, or —CO₂R₆ in which R₆ is hydrogen, alkyl, aryl, or aralkyl; and
n is 1 to 6;
or a pharmaceutically acceptable acid addition salt thereof; and
(c) 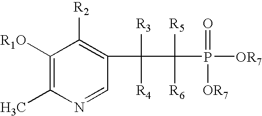
wherein,
R₁ is hydrogen or alkyl;
R₂ is —CHO, —CH₂OH, —CH₃ or —CO₂R₈ in which R₈ is hydrogen, alkyl, or aryl; or
R₂ is —CH₂—O-alkyl- in which alkyl is covalently bonded to the oxygen at the 3-position instead of R₁;
R₃ is hydrogen and R₄ is hydroxy, halo, alkoxy or alkylcarbonyloxy; or
R₃ and R₄ can be taken together to form ═O;
R₅ and R₆ are hydrogen; or
R₅ and R₆ are halo; and
R₇ is hydrogen, alkyl, aryl, aralkyl, or —CO₂R₈ in which R₈ is hydrogen, alkyl, aryl, or aralkyl;
or a pharmaceutically acceptable acid addition salt thereof.

7. The pharmaceutical composition according to claim 1, wherein the platelet aggregation inhibitor is a thromboxane A₂ inhibitor.

8. The pharmaceutical composition according to claim 7, wherein the thromboxane A₂ inhibitor is acetylsalicylic acid (ASA).

9. The pharmaceutical composition according to according to claim 1, wherein the platelet aggregation inhibitor is a glycoprotein IIb/IIIa inhibitor.

10. The pharmaceutical composition according to claim 9, wherein the platelet glycoprotein IIb/IIIa inhibitor is selected from the group consisting of eptifibatide, tirofiban, lamifiban, xemilofiban, orbofiban, sibrafiban, fradafiban, roxifiban, lotrafiban, and abciximab.

11. The pharmaceutical composition according to according to claim 1, wherein the platelet aggregation inhibitor is an adenosine diphosphate antagonist.

12. The pharmaceutical composition according to claim 11, wherein the adenosine diphosphate antagonist is selected from the group consisting of clopidogrel, ticlopidine, sulfinpyrazone, AZD6140, and AZD6933.

13. The pharmaceutical composition according to according to claim 1, wherein the platelet aggregation inhibitor is a cAMP phosphodiesterase inhibitor.

14. The pharmaceutical composition according to claim 13, wherein the cAMP phosophodiesterase inhibitor is selected from the group consisting of: dypyridamole, cilostazol, and pentoxifylline.

15. A method of inhibiting platelet aggregation in a mammal comprising administering a therapeutically effective dose of: (a) a compound selected from pyridoxal-5′-phosphate, a pyridoxal-5′-phosphate related compound, or a pharmaceutically acceptable salt thereof; and (b) a platelet aggregation inhibitor.

16. The method according to claim 15, wherein the pyridoxal-5′-phosphate related compound is selected from a group consisting of: pyridoxal, pyridoxal-5′-phosphate, pyridoxamine, a 3-acylated analogue of pyridoxal, a 3-acylated analogue of pyridoxal-4,5-aminal, a pyridoxine phosphate analogue, and a mixture thereof.

17. The method according to claim 16, wherein the compound is pyridoxal-5-phosphate.

18. The method according to claim 16, wherein the 3-acylated analogue of pyridoxal is: 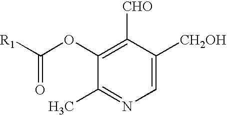
wherein,
R₁ is alkyl,
alkenyl,
in which alkyl or alkenyl
can be interrupted by nitrogen, oxygen, or sulfur, and
can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy; or
aryl, in which aryl can be substituted by alkyl, alkoxy, amino, hydroxy, halo, nitro, or alkanoyloxy
aryloxy,
arylthio, or
aralkyl, or a pharmaceutically acceptable acid addition salt thereof.

19. The method according to claim 16, wherein the 3-acylated analogue of pyridoxal-4,5-aminal is 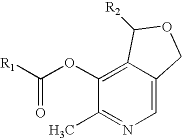
wherein,
R₁ is
alkyl,
alkenyl,
in which alkyl or alkenyl
can be interrupted by nitrogen, oxygen, or sulfur, and
can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy; or
aryl, in which aryl can be substituted by alkyl, alkoxy, amino, hydroxy, halo, nitro, or alkanoyloxy aryloxy,
arylthio, or
aralkyl; and
R₂ is a secondary amino group, or a pharmaceutically accpetable acid addition salt thereof.

20. The method according to claim 16, wherein the pyridoxine phosphate analogue is selected from a group comprising:
(a) 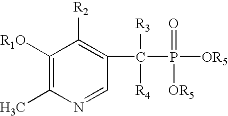
wherein,
R₁ is hydrogen or alkyl;
R₂ is —CH₂OH, —CH₃, —CO₂R₆ in which R₆ is hydrogen, alkyl, or aryl; or
R₂ is —CH₂—O-alkyl- in which alkyl is covalently bonded to the oxygen at the 3-position instead of R₁;
R₃ is hydrogen and R₄ is hydroxy, halo, alkoxy, alkylcarbonyloxy, alkylamino or arylamino; or
R₃ and R₄ are halo; and
R₅ is hydrogen, alkyl, aryl, aralkyl, or —CO₂R₇ in which R₇ is hydrogen, alkyl, aryl, or aralkyl;
or a pharmaceutically acceptable acid addition salt thereof;
(b) 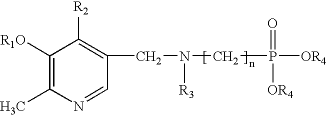
wherein,
R₁ is hydrogen or alkyl;
R₂ is —CHO, —CH₂OH, —CH₃ or —CO₂R₅ in which R₅ is hydrogen, alkyl, or aryl; or
R₂ is —CH₂—O-alkyl- (in which alkyl is covalently bonded to the oxygen at the 3-position instead of R₁);
R₃ is hydrogen, alkyl, aryl, or aralkyl;
R₄ is hydrogen, alkyl, aryl, aralkyl, or —CO₂R₆ in which R₆ is hydrogen, alkyl, aryl, or aralkyl; and
n is 1 to 6;
or a pharmaceutically acceptable acid addition salt thereof; and
(c) 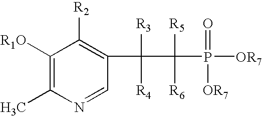
wherein,
R₁ is hydrogen or alkyl;
R₂ is —CHO, —CH₂OH, —CH₃ or —CO₂R₈ in which R₈ is hydrogen, alkyl, or aryl; or
R₂ is —CH₂—O-alkyl- in which alkyl is covalently bonded to the oxygen at the 3-position instead of R₁;
R₃ is hydrogen and R₄ is hydroxy, halo, alkoxy or alkylcarbonyloxy; or
R₃ and R₄ can be taken together to form ═O;
R₅ and R₆ are hydrogen; or
R₅ and R₆ are halo; and
R₇ is hydrogen, alkyl, aryl, aralkyl, or —CO₂R₈ in which R₈ is hydrogen, alkyl, aryl, or aralkyl;
or a pharmaceutically acceptable acid addition salt thereof.

21. The method according to claim 15, wherein the platelet aggregation inhibitor is a thromboxane A₂ inhibitor.

22. The method according to claim 21, wherein the thromboxane A₂ inhibitor is acetylsalicylic acid (ASA).

23. The method according to claim 15, wherein the platelet aggregation inhibitor is a glycoprotein IIb/IIIa inhibitor.

24. The method according to claim 23, wherein the platelet glycoprotein IIb/IIIa inhibitor is eptifibatide.

25. The method according to claim 15, wherein the platelet aggregation inhibitor is an adenosine diphosphate antagonist.

26. The method according to claim 25, wherein the adenosine diphosphate antagonist is selected from the group consisting of: clopidogrel, ticlopidine, AZD6140, and AZD6933.

27. The method according to claim 15, wherein the platelet aggregation inhibitor is a cAMP phosphodiesterase inhibitor.

28. The method according to claim 27, wherein the cAMP phosphodiesterase inhibtior is selected from a group consisting of dypyridamole, cilostazol, and pentoxifylline.

29. The method according to claim 15, wherein the dose of the pyridoxal-5′-phosphate or pyridoxal-5′-phosphate related compound is between 0.1 to 50 mg/kg per day.

30. The method according to claim 15, wherein the dose of the pyridoxal-5′-phosphate or pyridoxal-5′-phosphate related compound is between 1 to 5 mg/kg per day.

31. A method of treating a mammalian patient at risk of a cardiovascular disease comprising administering a therapeutically effective dose of the pharmaceutical composition according to claim 1.

32. The method according to claim 31, wherein the cardiovascular disease is selected from a group comprising: congestive heart failure, myocardial ischemia, arrhythmia, myocardial infarction, ischemic stroke, hemorrhagic stroke, coronary artery disease, hypertension (high blood pressure), atherosclerosis (clogging of the arteries), aneurysm, peripheral artery disease, thrombophlebitis (vein inflammation), diseases of the heart lining, diseases of the heart muscle, carditis, congestive heart failure, endocarditis, ischemic heart disease, valvular heart disease (malfunction of a valve or valves in the blood vessels of the heart), peripheral vascular disease, ischemic injury, Kawazaki disease, arteriosclerosis (hardening of the arteries), deep vein thrombosis, and acute coronary syndrome.

33. The method according to claim 31, wherein the dose of the pyridoxal-5′-phosphate or pyridoxal-5′-phosphate related compound is between 0.1 to 50 mg/kg per day.

34. The method according to claim 31, wherein the dose of the pyridoxal-5′-phosphate or pyridoxal-5′-phosphate related compound is between 1 to 5 mg/kg per day.

35. A method of treating a mammal having a disease which arises from thrombotic and prothrombotic states in which the coagulation cascade is activated, comprising administering a therapeutically effective dose of the pharmaceutical composition according to claim 1.

36. The method according to claim 35, wherein the disease is selected from a group consisting of: deep vein thrombosis, disseminated intravascular coagulopathy, and pulmonary embolism.

37. The method according to claim 35, wherein the dose of the pyridoxal-5′-phosphate or pyridoxal-5′-phosphate related compound is between 0.1 to 50 mg/kg per day.

38. The method according to claim 35, wherein the dose of the pyridoxal-5′-phosphate or pyridoxal-5′-phosphate related compound is between 1 to 5 mg/kg per day.

39. A method of treating a mammalian patient at risk of cerebrovascular disease comprising administering a therapeutically effective dose of the pharmaceutical composition according to claim 1.

40. The method according to claim 39, wherein the cerebrovascular disease is selected from a group consisting of: cerebral ischemia, cerebral hemorrhage, ischemic stroke, and hemorrhagic stroke.

41. The method according to claim 39, wherein the dose of the pyridoxal-5′-phosphate or pyridoxal-5′-phosphate related compound is between 0.1 to 50 mg/kg per day.

42. The method according to claim 39, wherein the dose of pyridoxal-5′-phosphate or pyridoxal-5′-phosphate related compound is between 1 to 5 mg/kg per day.

43. A method for of treating a mammalian patient at risk of a cardiovascular disease comprising administering a therapeutically effective dose of: (a) a compound selected from pyridoxal-5′-phosphate, a pyridoxal-5′-phosphate related compound, or a pharmaceutically acceptable salt thereof; and (b) a platelet aggregation inhibitor.

44. The method according to claim 43, wherein the cardiovascular disease is selected from a group comprising: congestive heart failure, myocardial ischemia, arrhythmia, myocardial infarction, ischemic stroke, hemorrhagic stroke, coronary artery disease, hypertension (high blood pressure), atherosclerosis (clogging of the arteries), aneurysm, peripheral artery disease, thrombophlebitis (vein inflammation), diseases of the heart lining, diseases of the heart muscle, carditis, congestive heart failure, endocarditis, ischemic heart disease, valvular heart disease (malfunction of a valve or valves in the blood vessels of the heart), peripheral vascular disease, ischemic injury, Kawazaki disease, arteriosclerosis (hardening of the arteries), deep vein thrombosis, and acute coronary syndrome.

45. The method according to claim 43, wherein the cardiovascular disease is myocardial infarction, transient ischemic attack or ischemic stroke and wherein the platelet aggregation inhibitor is acetylsalicylic acid (ASA) and the compound is pyridoxal-5′-phosphate.

46. The method according to claim 45, wherein the therapeutically effective dose of the acetylsalicylic acid is between 5 and 500 mg/day.

47. The method according to claim 45, wherein the therapeutically effective dose of the acetylsalicylic acid is between 30 and 81 mg/day.

48. The method according to claim 45, wherein the therapeutically effective dose of the acetylsalicylic acid is between 75 and 81 mg/day.

49. The method according to claim 43, wherein the cardiovascular disease is acute coronary syndrome and wherein the platelet aggregation inhibitor is eptifibatide and the compound is pyridoxal-5′-phosphate.

50. The method according to claim 49, wherein therapeutically effective dose of eptifibatide is between 30 and 500 μg/kg.

51. The method according to claim 49, wherein the eptifibatide is administered intravenously.

52. The method according to claim 49, wherein the eptifibatide is administered as a bolus injection of 180 μg/kg following diagnosis of acute coronary syndrome and is then administered as a continuous IV infusion of between 0.1 to 5 μg/kg/min for up to 72 hours.

53. The method according to claim 52, wherein the eptifibatide is administered as a continuous IV infusion of 2 μg/kg/min.

54. The method according to claim 43, wherein the platelet aggregation inhibitor is clopidogrel and the compound is pyridoxal-5′-phosphate.

55. The method according to claim 54, wherein the therapeutically effective dose of clopidogrel is between 10 and 1000 mg per day.

56. The method according to claim 54, wherein the therapeutically effective dose of clopidogrel is between 75 and 150 mg per day.

57. The method according to claim 54, wherein the therapeutically effective dose of clopidogrel is 75 mg per day.

58. A method for of treating a mammalian patient undergoing a cardiovascular surgical intervention comprising administering a therapeutically effective dose of (a) a compound selected from pyridoxal-5′-phosphate, a pyridoxal-5′-phosphate related compound or a pharmaceutically acceptable salt thereof and (b) a platelet aggregation inhibitor, prior to the surgical intervention or following the surgical intervention.

59. The method according to claim 58, wherein the surgical intervention is percutaneous coronary intervention and the platelet aggregation inhibitor is eptifibatide.

60. The method according to claim 59, wherein the therapeutically effective dose of eptifibatide is between 30 to 500 μg/kg.

61. The method according to claim 59, wherein the eptifibatide is administered as a bolus IV injection of 135 μg/kg immediately prior to the percutaneous coronary intervention and as a continuous IV infusion of between 0.1 and 5 μg/kg/min following for between 20 to 24 hours following the percutaneous coronary intervention.

62. The method according to claim 58, wherein the surgical intervention is the placement of a coronary stent and the platelet aggregation inhibitor is eptifibatide.

63. The method according to claim 62, wherein the eptifibatide is administered as a first bolus IV injection of 180 μg/kg immediately prior to the placement of the coronary stent intervention, as a continuous IV infusion of between 0.1 and 5 μg/kg/min following for 10 minutes following placement of the coronary stent, and then as a second bolus IV injection of 180 μg/kg.

64. The method according to claim 63, wherein the eptifibatide is administered as a continuous IV infusion of 2 μg/kg/min.

65. The method according to claim 63, wherein following the second bolus IV injection of the eptifibatide, a continuous IV infusion of between 0.1 and 5 μg/kg/min of the eptifibatide is administered for between 18 and 24 hours.

66. The method according to claim 58, wherein platelet aggregation inhibitor is clopidogrel.

67. The method according to claim 66, wherein the therapeutically effective dosage is between 300 and 500 mg and wherein the clopidogrel is administered prior to the surgical intervention.

68. The method according to claim 66, wherein the therapeutically effective dosage is between 300 and 350 mg and wherein the clopidogrel is administered prior to the surgical intervention.

69. The method according to claim 66, wherein the therapeutically effective dosage is 300 mg and wherein the clopidogrel is administered prior to the surgical intervention.

70. The method according to claim 58, wherein the surgical intervention is a coronary artery bypass graft or a percutaneous coronary intervention and the platelet aggregation inhibitor is acetylsalicylic acid.

71. The method according to claim 70, wherein the therapeutically effective dosage is 325 mg and wherein the acetylsalicylic acid is administered following the surgical intervention.

72. The method according to claim 70, wherein the therapeutically effective dosage is 325 mg and wherein the acetylsalicylic acid is administered daily for 3 days following the surgical intervention.
